(19) United States
(12) Patent Application Publication
(10) Pub. No.: US 2005/0059818 A1
(43) Pub. Date: Mar. 17, 2005

(54) Polymorph of a pharmaceutical

(76) Inventors: Richard W. Duerst, Lake Bluff, IL (US); Devalina Law, Libertyville, IL (US); Xiaochun Lou, Long Grove, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/661,148

(22) Filed: Sep. 12, 2003

(51) Int. Cl. C07D 501/14

(52) U.S. Cl. 540/222

(57) ABSTRACT

The present invention relates to novel crystalline polymorphs of 7-[2-(2-aminothiazol-4-yl)-2-hydroxyiminoacetamide]-3-vinyl-3-cephem-4-carboxylic acid (syn isomer), methods for their preparation, and pharmaceutical compositions comprising the novel crystalline polymorphs.

### TECHNICAL FIELD

The present invention relates to novel crystalline polymorphs of 7-[2-(2-aminothiazol-4-yl)-2-hydroxyiminoacetamide]-3-vinyl-3-cephem-4-carboxylic acid (syn isomer), methods for their preparation, and pharmaceutical compositions comprising the novel crystalline polymorphs.

### BACKGROUND OF THE INVENTION

The antimicrobial agent 7-[2-(2-aminothiazol-4-yl)-2-hydroxyiminoacetamido]-3-vinyl-3-cephem-4-carboxylic acid (syn isomer) (hereinafter referred to as “Cefdinir”) is a semi-synthetic oral antibiotic in the cephalosporin family. Cefdinir is active against a very wide spectrum of bacteria, including Staphylococcus aureus, Streptococcus pneumoniae, Streptococcus pogenes, Hemophilus influenzae, Moraxella catarrhalis, E. coli, Klebsiella, and Proteus mirabilis. The preparation of this agent was first disclosed in U.S. Pat. No. 4,559,334, issued Dec. 17, 1985, which is hereby incorporated by reference in its entirety.

A novel crystalline form of Cefdinir (originally referred to as “Crystal A”, herein referred to as “Form I”) was first disclosed in U.S. Pat. No. 4,935,507, issued Jun. 19, 1990, which is hereby incorporated by reference in its entirety. While this polymorph does overcome several of the problems associated with the amorphous form, the formation of additional new polymorphs can provide further advantages such as increased stability.

It has now been unexpectedly discovered that Cefdinir can be prepared as a new crystalline polymorph which is termed Form II.

### BRIEF DESCRIPTION OF THE FIGURE

FIG. 1 is a representative powder X-ray diffraction pattern of the Form I crystalline polymorph of Cefdinir.

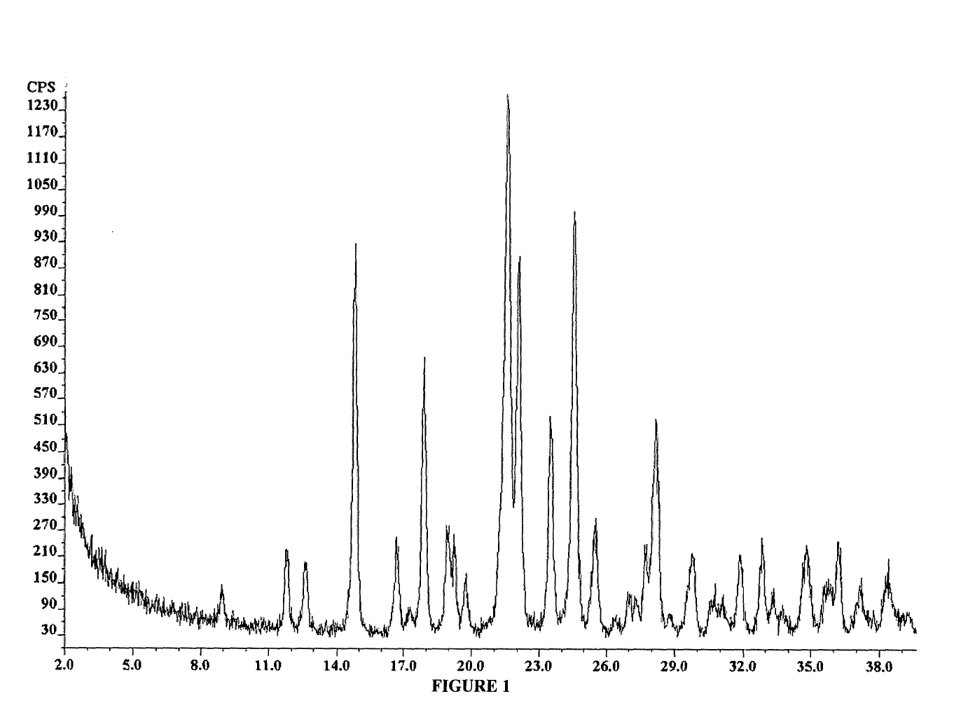

FIG. 2 is a representative powder X-ray diffraction pattern of the Form II crystalline polymorph of Cefdinir.

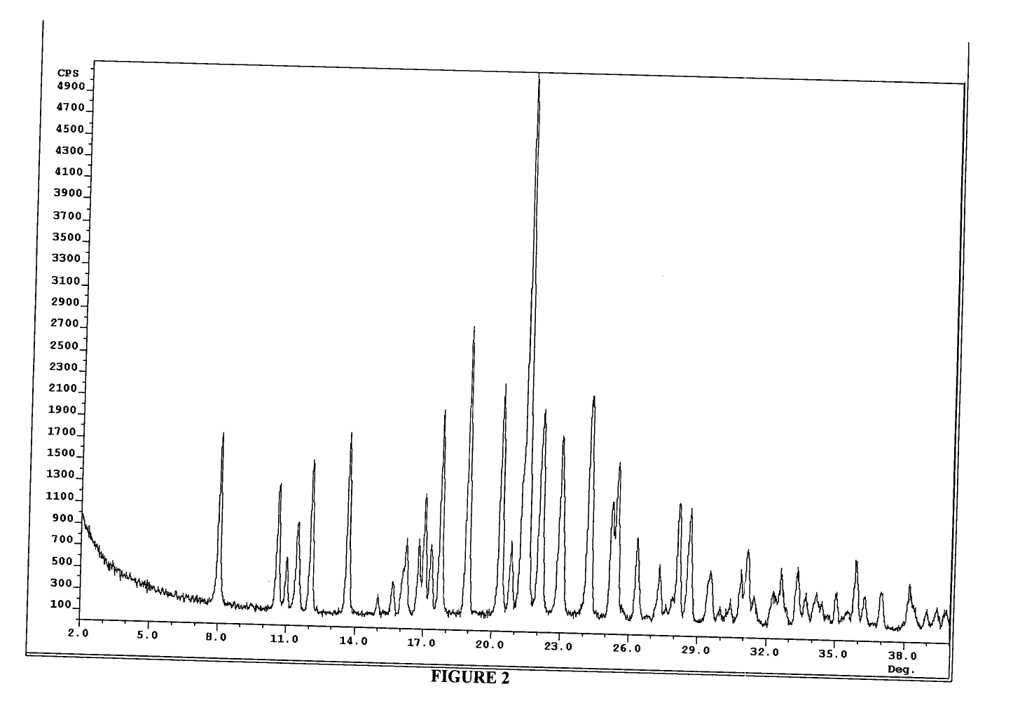

FIG. 3 is the infrared spectrum of the Form I crystalline polymorph of Cefdinir.

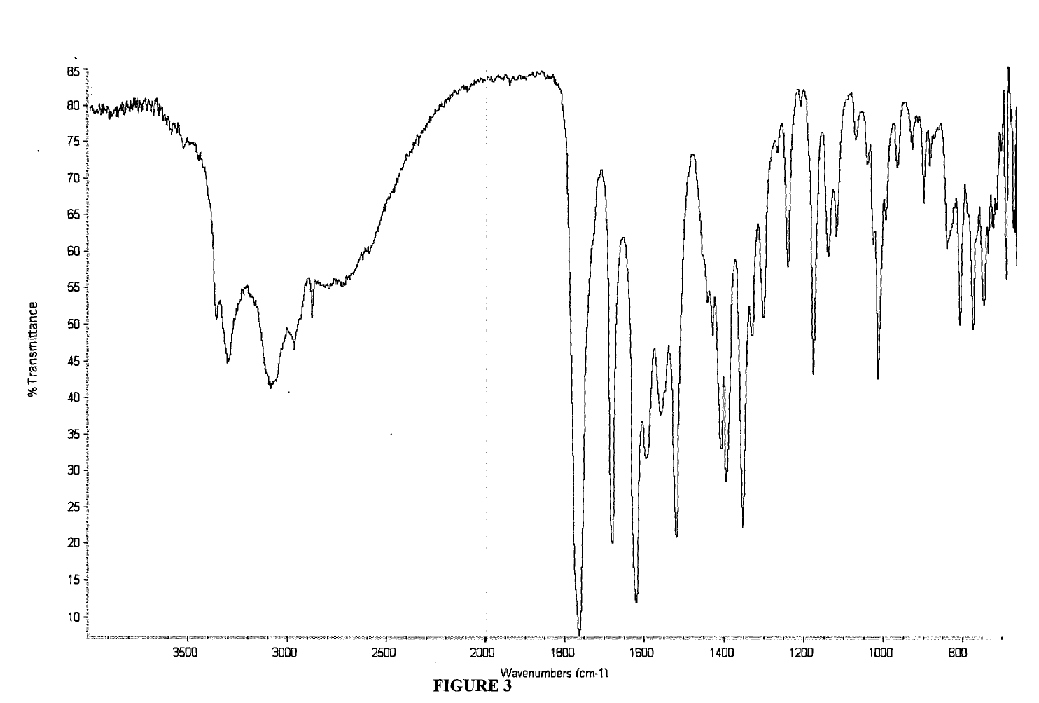

FIG. 4 is the infrared spectrum of the Form II crystalline polymorph of Cefdinir.

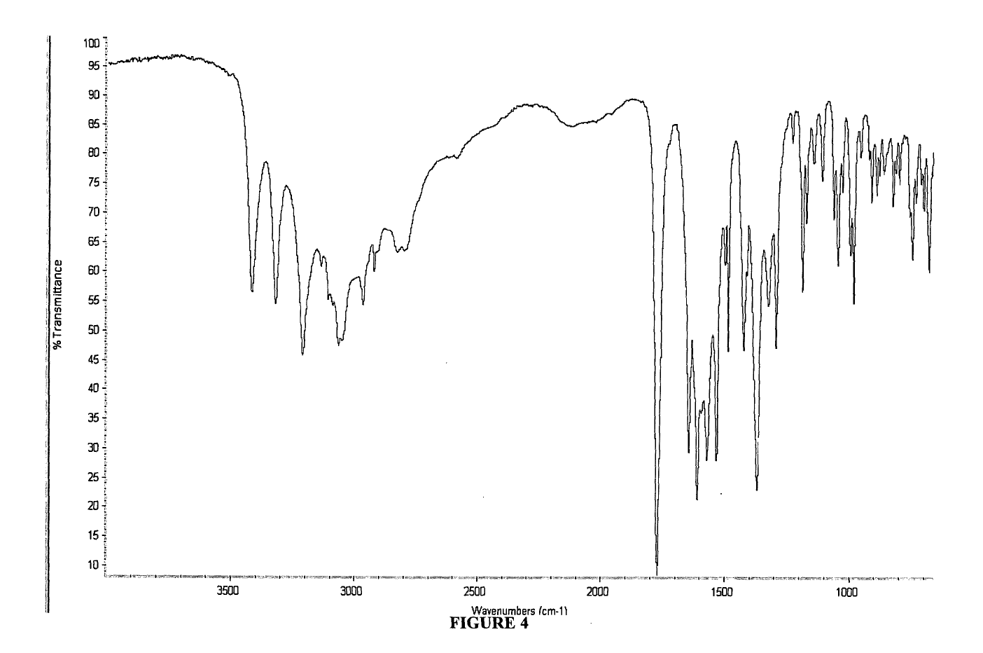

FIG. 5 is the TGA of the Form II crystalline polymorph of Cefdinir.

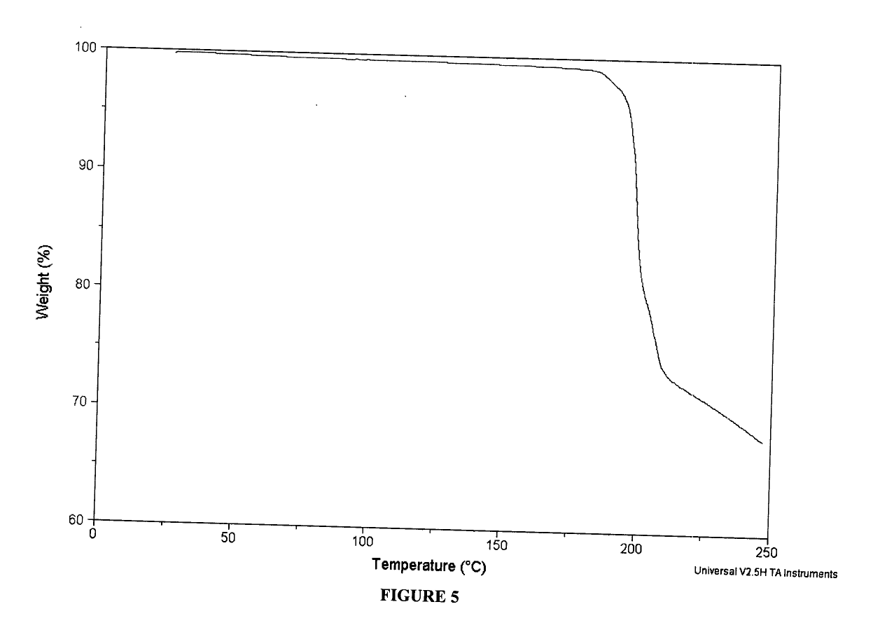

### SUMMARY OF THE INVENTION

The present invention describes a novel crystalline polymorphs of Cefdinir. For the sake of identification, this crystalline polymorph is designated as the Form II crystalline polymorph of Cefidinir.

In its principle embodiment the present invention describes a crystalline polymorph of Cefdinir with characteristic peaks in the powder X-ray diffraction pattern at values of two theta of 8.1±0.1°, 10.7±0.1°, 12.1±0.1°, 13.7±0.1°, 17.8±0.1°, 19.0±0.1°, 20.4±0.1°, 21.5±0.1°, 22.2±0.1°, 23.0±0.1°, 24.3±0.1°, and 25.5±0.1°.

In another embodiment the present invention describes a crystalline polymorph of Cefdinir prepared by a process comprising suspending crystalline Form I of Cefdinir (preferably about 300 mg) in a solvent for a period of time (preferably about 1 to about 8 weeks) followed by isolating the desired polymorph. Preferably this process is conducted at about 20° C. to about 40° C., most preferably at about 23° C. Preferred solvents are water, ethanol, methanol, propanol, isopropanol, acetonitrile, formamide, N-methylpyrrolidinone, N,N-dimethylformamide, triethylamine, diisopropylethylamine, toluene, xylene, mesitylene, ethyl acetate, isopropyl acetate, tetrahydrofuran, dioxane, diethyl ether, methyl tert-butyl ether, dichloromethane, chloroform, carbon tetrachloride, hexane, pentane, heptane, acetone, methyl ethyl ketone, dimethylsulfoxide, pyridine, nitromethane, and mixtures thereof. More preferred solvents are water, ethanol, acetonitrile, formamide, N-methylpyrroldinone, triethylamine, toluene, ethyl acetate, tetrahydrofuran, dioxane, dichloromethane, hexane, acetone, methyl ethyl ketone, dimethylsulfoxide, pyridine, nitromethane, 1:1 water/ethanol, 1:1 water/acetonitrile, and 1:1 water/acetone. A most preferred solvent is pyridine.

In another embodiment the present invention describes a process for the preparation of the crystalline polymorph of claim 1 comprising suspending Form I of Cefdinir in a solvent, then isolating the desired polymorph. Preferably this process is conducted at about 20° C. to about 40° C., most preferably at about 23° C. Preferred solvents are water, ethanol, methanol, propanol, isopropanol, acetonitrile, formamide, N-methylpyrrolidinone, N,N-dimethylformamide, triethylamine, diisopropylethylamine, toluene, xylene, mesitylene, ethyl acetate, isopropyl acetate, tetrahydrofuran, dioxane, diethyl ether, methyl tert-butyl ether, dichloromethane, chloroform, carbon tetrachloride, hexane, pentane, heptane, acetone, methyl ethyl ketone, dimethylsulfoxide, pyridine, nitromethane, and mixtures thereof. More preferred solvents are water, ethanol, acetonitrile, formamide, N-methylpyrroldinone, triethylamine, toluene, ethyl acetate, tetrahydrofuran, dioxane, dichloromethane, hexane, acetone, methyl ethyl ketone, dimethylsulfoxide, pyridine, nitromethane, 1:1 water/ethanol, 1:1 water/acetonitrile, and 1:1 water/acetone. A most preferred solvent is pyridine.

In another embodiment the present invention describes a pharmaceutical composition comprising crystal Form II of Cefdinir in combination with a pharmaceutically acceptable carrier.

### DETAILED DESCRIPTION OF THE INVENTION

Powder X-ray diffraction was performed using an XDS-2000/X-ray diffractometer equipped with a 2 kW normal focus X-ray tube and a Peltier cooled germanium solid-state detector (Scintag Inc., Sunnyvale, Calif.). The data was processed using DMSNT software (version 1.37). The X-ray source was a copper filament operated at 45 kV and 40 mA. The alignment of the goniometer was checked daily using a Corundum standard. The sample was placed in a thin layer onto a zero background plate, and continuously scanned at a rate of 2° two-theta per minute over a range of 2 to 40° two-theta.

Characteristic powder X-ray diffraction pattern peak positions are reported for polymorphs in terms of the angular positions (two theta) with an allowable variability of ±0.1°. This allowable variability is specified by the U.S. Pharmacopeia, pages 1843-1884 (1995). The variability of ±0.1° is intended to be used when comparing two powder X-ray diffraction patterns. In practice, if a diffraction pattern peak from one pattern is assigned a range of angular positions (two theta) which is the measured peak position ±0.1° and if those ranges of peak positions overlap, then the two peaks are considered to have the same angular position (two theta). For example, if a diffraction pattern peak from one pattern is determined to have a peak position of 5.2°, for comparison purposes the allowable variability allows the peak to be assigned a position in the range of 5.1°-5.3°. If a comparison peak from the other diffraction pattern is determined to have a peak position of 5.3°, for comparison purposes the allowable variability allows the peak to be assigned a position in the range of 5.2°-5.4°. Because there is overlap between the two ranges of peak positions (i.e., 5.1°-5.3° and 5.2°-5.4°) the two peaks being compared are considered to have the same angular position (two theta).

Transmission infrared spectroscopy of the solids were obtained using a Fourier-transform infrared spectrometer (Nicolet Magna 750 FT-IR Spectrometer, Nicolet Instrument Corporation, Madison, Wis.) equipped with a Nicolet NIC-PLAN microscope. The microscope had an MCT-A liquid nitrogen cooled detector. The sample was rolled on a 13 mm×1 mm BaF₂ disc sample holder; 64 scans were collected at 4 cm⁻¹ resolution.

Thermogravimetric analysis was performed in TA Instruments TG2950 (TA Instruments, New Castle, Del.). The samples were scanned at 10° C./minute with a dry nitrogen purge at 60 mL/minute.

In accordance with methods of treatment and pharmaceutical compositions of the invention, the compounds can be administered alone or in combination with other agents. When using the compounds, the specific therapeutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidently with the compound used. The compounds can be administered orally, parenterally, intranasally, rectally, vaginally, or topically in unit dosage formulations containing carriers, adjuvants, diluents, vehicles, or combinations thereof. The term “parenteral” includes infusion as well as subcutaneous, intravenous, intramuscular, and intrasternal injection.

Parenterally administered aqueous or oleaginous suspensions of the compounds can be formulated with dispersing, wetting, or suspending agents. The injectable preparation can also be an injectable solution or suspension in a diluent or solvent. Among the acceptable diluents or solvents employed are water, saline, Ringer's solution, buffers, monoglycerides, diglycerides, fatty acids such as oleic acid, and fixed oils such as monoglycerides or diglycerides.

The effect of parenterally administered compounds can be prolonged by slowing their absorption. One way to slow the absorption of a particular compound is administering injectable depot forms comprising suspensions of poorly soluble crystalline or otherwise water-insoluble forms of the compound. The rate of absorption of the compound is dependent on its rate of dissolution which, in turn, is dependent on its physical state. Another way to slow absorption of a particular compound is administering injectable depot forms comprising the compound as an oleaginous solution or suspension. Yet another way to slow absorption of a particular compound is administering injectable depot forms comprising microcapsule matrices of the compound trapped within liposomes, or biodegradable polymers such as polylactide-polyglycolide, polyorthoesters or polyanhydrides. Depending on the ratio of drug to polymer and the composition of the polymer, the rate of drug release can be controlled.

Transdermal patches can also provide controlled delivery of the compounds. The rate of absorption can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise excipients such as sucrose, lactose, starch, microcrystalline cellulose, mannitol, talc, silicon dioxide, polyvinylpyrrolidone, sodium starch glycolate, magnesium stearate, etc. Capsules, tablets and pills can also comprise buffering agents, and tablets and pills can be prepared with enteric coatings or other release-controlling coatings. Powders and sprays can also contain excipients such as talc, silicon dioxide, sucrose, lactose, starch, or mixtures thereof. Sprays can additionally contain customary propellants such as chlorofluorohydrocarbons or substitutes thereof.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents. Liquid dosage forms may also be contained within soft elastic capsules.

Topical dosage forms include ointments, pastes, creams, lotions, gels, powders, solutions, sprays, inhalants, and transdermal patches. The compound is mixed, if necessary under sterile conditions, with a carrier and any needed preservatives or buffers. These dosage forms can also include excipients such as animal and vegetable fats, oils, waxes, paraffins, starch, tragacanth, cellulose derivatives, polyethylene glycols, silicones, bentonites, talc and zinc oxide, or mixtures thereof. Suppositories for rectal or vaginal administration can be prepared by mixing the compounds with a suitable non-irritating excipient such as cocoa butter or polyethylene glycol, each of which is solid at ordinary temperature but fluid in the rectum or vagina. Ophthalmic formulations comprising eye drops, eye ointments, powders, and solutions are also contemplated as being within the scope of this invention.

The following examples will serve to further illustrate the preparation of the novel crystal forms. Form I of Cefdinir was prepared according to the procedure described in U.S. Pat. No. 4,935,507, issued Jun. 19, 1990.

### EXAMPLE 1

### Preparation of Novel Cefdinir Polymorph from Water

The solubility of Cefdinir Form I in water was determined. A suspension of Cefdinir Form I (300 mg in excess of the determined solubility) in 4 mL of water was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 2

### Preparation of Novel Cefdinir Polymorph from Ethanol

The solubility of Cefdinir Form I in ethanol was determined. A suspension of Cefdinir Form I (300 mg in excess of the solubility) in 4 mL of ethanol was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 3

### Preparation of Novel Cefdinir Polymorph from Acetonitrile

The solubility of Cefdinir Form I in acetonitrile was determined. A suspension of Cefdinir Form I (300 mg in excess of the solubility) in 4 mL of acetonitrile was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 4

### Preparation of Novel Cefdinir Polymorph from Formamide

The solubility of Cefdinir Form I in formamide was determined. A suspension of Cefdinir Form I (300 mg in excess of the solubility) in 4 mL of formamide was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 5

### Preparation of Novel Cefdinir Polymorph from N-methylpyrrolidinone

The solubility of Cefdinir Form I in N-methylpyrrolidinone was determined. A suspension of Cefdinir Form I (300 mg in excess of the solubility) in 4 mL of N-methylpyrrolidinone was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 6

### Preparation of Novel Cefdinir Polymorph from Triethylamine

The solubility of Cefdinir Form I in triethylamine was determined. A suspension of Cefdinir Form I (300 mg in excess of the solubility) in 4 mL of triethylamine was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 7

### Preparation of Novel Cefdinir Polymorph from Toluene

The solubility of Cefdinir Form I in toluene was determined. A suspension of Cefdinir Form I (300 mg in excess of the solubility) in 4 mL of toluene was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 8

### Preparation of Novel Cefdinir Polymorph from Ethyl Acetate

The solubility of Cefdinir Form I in ethyl acetate was determined. A suspension of Cefdinir Form I (300 mg in excess of the solubility) in 4 mL of ethyl acetate was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 9

### Preparation of Novel Cefdinir Polymorph from Tetrahydrofuran

The solubility of Cefdinir Form I in tetrahydrofuran was determined. A suspension of Cefdinir Form I (300 mg in excess of the solubility) in 4 mL of tetrahydrofuran was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 10

### Preparation of Novel Cefdinir Polymorph from Dioxane

The solubility of Cefdinir Form I in dioxane was determined. A suspension of Cefdinir Form I (300 mg in excess of the solubility) in 4 mL of dioxane was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 11

### Preparation of Novel Cefdinir Polymorph from Dichloromethane

The solubility of Cefdinir Form I in dichloromethane was determined. A suspension of Cefdinir Form I (300 mg in excess of the solubility) in 4 mL of dichloromethane was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 12

### Preparation of Novel Cefdinir Polymorph from Hexane

The solubility of Cefdinir Form I in hexane was determined. A suspension of Cefdinir Form I (300 mg in excess of the solubility) in 4 mL of hexane was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 13

### Preparation of Novel Cefdinir Polymorph from Acetone

The solubility of Cefdinir Form I in acetone was determined. A suspension of Cefdinir Form I (300 mg in excess of the solubility) in 4 mL of acetone was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 14

### Preparation of Novel Cefdinir Polymorph from Methyl Ethyl Ketone

The solubility of Cefdinir Form I in methyl ethyl ketone was determined. A suspension of Cefdinir Form I (300 mg in excess of the solubility) in 4 mL of methyl ethyl ketone was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 15

### Preparation of Novel Cefdinir Polymorph from Dimethylsulfoxide

The solubility of Cefdinir Form I in dimethylsulfoxide was determined. A suspension of Cefdinir Form I (300 mg in excess of the solubility) in 4 mL of dimethylsulfoxide was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 16

### Preparation of Novel Cefdinir Polymorph from Pyridine

The solubility of Cefdinir Form I in pyridine was determined. A suspension of Cefdinir Form I (300 mg in excess of the solubility) in 4 mL of pyridine was allowed to stand at room temperature. After 1 week, the solid from the suspension was separated and the saturated solution was reserved. The powder X-ray diffraction pattern of the moist solid was generated and the solid was returned to the reserved solution.

### EXAMPLE 17

### Preparation of Novel Cefdinir Polymorph from Nitromethane

The solubility of Cefdinir Form I in nitromethane was determined. A suspension of Cefdinir Form I (300 mg in excess of the solubility) in 4 mL of nitromethane was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 18

### Preparation of Novel Cefdinir Polymorph from 1:1 Water/Ethanol

The solubility of Cefdinir Form I in 1:1 water/ethanol was determined. A suspension of Cefdinir Form I (300 mg in excess of the solubility) in 4 mL of 1:1 water/ethanol was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 19

### Preparation of Novel Cefdinir Polymorph from 1:1 Water/Acetonitrile

The solubility of Cefdinir Form I in 1:1 water/acetonitrile was determined. A suspension of Cefdinir Form I (300 mg in excess of the solubility) in 4 mL of 1:1 water/acetonitrile was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

### EXAMPLE 20

### Preparation of Novel Cefdinir Polymorph from 1:1 Water/Acetone

The solubility of Cefdinir Form I in 1:1 water/acetone was determined. A suspension of Cefdinir Form I (300 mg in excess of the solubility) in 4 mL of 1:1 water/acetone was allowed to stand at room temperature. After 1 week, the solid from the suspension is separated and the saturated solution is reserved. The powder X-ray diffraction pattern of the moist solid is generated and the solid is returned to the reserved solution. If a difference is seen between the newly generated diffraction pattern and that of the original Cefdinir the suspension is examined again at weeks 2, 4, and 8, or until it is determined that the suspended solid has been completely transformed into the new phase. At this time the new phase is characterized by powder X-ray diffraction, thermal methods (DSC, THA, HSM), and spectroscopic methods (mid IR, NIR) to determine whether the new phase is a solvate or a polymorph. If the new phase is a solvate, the desolvated phase is isolated in an attempt to determine the stoichiometry of the solvate, the existence of isomorphs, and the existence of a desolvated phase having a new crystal lattice.

The foregoing is merely illustrative of the invention and is not intended to limit the invention to the disclosed embodiments. Variations and changes which are obvious to one skilled in the art are intended to be within the scope and nature of the invention which are defined in the appended claims.

1. A crystalline polymorph of Cefdinir with characteristic peaks in the powder X-ray diffraction pattern at values of two theta of 8.1±0.1°, 10.7±0.1°, 12.1±0.1°, 13.7±0.1°, 17.8±0.1°, 19.0±0.1°, 20.4±0.1°, 21.5±0.1°, 22.2±0.1°, 23.0±0.1°, 24.3±0.1°, and 25.5±0.1°.

2. A crystalline polymorph of Cefdinir prepared by a process comprising:
(a) suspending Form I of Cefdinir in a solvent;
(b) isolating the desired polymorph from the suspension of step (a).

3. The crystalline polymorph of claim 2 wherein the solvent is water.

4. The crystalline polymorph of claim 2 wherein the solvent is ethanol.

5. The crystalline polymorph of claim 2 wherein the solvent is acetonitrile.

6. The crystalline polymorph of claim 2 wherein the solvent is formamide.

7. The crystalline polymorph of claim 2 wherein the solvent is N-methylpyrroldinone.

8. The crystalline polymorph of claim 2 wherein the solvent is triethylamine.

9. The crystalline polymorph of claim 2 wherein the solvent is toluene.

10. The crystalline polymorph of claim 2 wherein the solvent is ethyl acetate.

11. The crystalline polymorph of claim 2 wherein the solvent is tetrahydrofuran.

12. The crystalline polymorph of claim 2 wherein the solvent is dioxane.

13. The crystalline polymorph of claim 2 wherein the solvent is dichloromethane.

14. The crystalline polymorph of claim 2 wherein the solvent is hexane.

15. The crystalline polymorph of claim 2 wherein the solvent is acetone.

16. The crystalline polymorph of claim 2 wherein the solvent is methyl ethyl ketone.

17. The crystalline polymorph of claim 2 wherein the solvent is dimethylsulfoxide.

18. The crystalline polymorph of claim 2 wherein the solvent is pyridine.

19. The crystalline polymorph of claim 2 wherein the solvent is nitromethane.

20. The crystalline polymorph of claim 2 wherein the solvent is a 1:1 mixture of water and ethanol.

21. The crystalline polymorph of claim 2 wherein the solvent is a 1:1 mixture of water and acetonitrile.

22. The crystalline polymorph of claim 2 wherein the solvent is a 1:1 mixture of water and acetone.

23. The crystalline polymorph of claim 1 wherein the suspension of step (a) has about 300 mg of Form I of Cefdinir.

24. The crystalline polymorph of claim 2 wherein step (a) is conducted at about 20° C. to about 40° C.

25. The crystalline polymorph of claim 2 wherein step (a) is conducted at about 23° C.

26. The crystalline polymorph of claim 2 wherein step (a) is conducted for about 1 to about 8 weeks.

27. A process for preparing a crystalline polymorph of Cefdinir, the process comprising:
(a) suspending Form I of Cefdinir in a solvent;
(b) isolating the desired polymorph from the suspension of step (a).

28. The crystalline polymorph of claim 27 wherein the solvent is water.

29. The crystalline polymorph of claim 27 wherein the solvent is ethanol.

30. The crystalline polymorph of claim 27 wherein the solvent is acetonitrile.

31. The crystalline polymorph of claim 27 wherein the solvent is formamide.

32. The crystalline polymorph of claim 27 wherein the solvent is N-methylpyrroldinone.

33. The crystalline polymorph of claim 27 wherein the solvent is triethylamine.

34. The crystalline polymorph of claim 27 wherein the solvent is toluene.

35. The crystalline polymorph of claim 27 wherein the solvent is ethyl acetate.

36. The crystalline polymorph of claim 27 wherein the solvent is tetrahydrofuran.

37. The crystalline polymorph of claim 27 wherein the solvent is dioxane.

38. The crystalline polymorph of claim 27 wherein the solvent is dichloromethane.

39. The crystalline polymorph of claim 27 wherein the solvent is hexane.

40. The crystalline polymorph of claim 27 wherein the solvent is acetone.

41. The crystalline polymorph of claim 27 wherein the solvent is methyl ethyl ketone.

42. The crystalline polymorph of claim 27 wherein the solvent is dimethylsulfoxide.

43. The crystalline polymorph of claim 27 wherein the solvent is pyridine.

44. The crystalline polymorph of claim 27 wherein the solvent is nitromethane.

45. The crystalline polymorph of claim 27 wherein the solvent is a 1:1 mixture of water and ethanol.

46. The crystalline polymorph of claim 27 wherein the solvent is a 1:1 mixture of water and acetonitrile.

47. The crystalline polymorph of claim 27 wherein the solvent is a 1:1 mixture of water and acetone.

48. The process of claim 27 wherein the suspension of step (a) has about 300 mg of Form I of Cefdinir.

49. The process of claim 27 wherein step (a) is conducted at about 20° C. to about 40° C.

50. The process of claim 27 wherein step (a) is conducted at about 23° C.

51. The process of claim 27 wherein step (a) is conducted for about 1 to about 8 weeks.

52. A pharmaceutical composition comprising the crystalline polymorph of claim 1 in combination with a pharmaceutically acceptable carrier.
